(19) United States
(12) Patent Application Publication
(10) Pub. No.: US 2001/0043928 A1
(43) Pub. Date: Nov. 22, 2001

(54) Serum preparations for inhibiting retroviruses and tumor growth

(76) Inventor: Mark E. Howard, Everett, WA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/790,272

(22) Filed: Feb. 21, 2001

Related U.S. Application Data
(63) Continuation of application No. 08/434,438, filed on May 3, 1995, now abandoned.

(51) Int. Cl. A61K 39/395; A61K 35/16; A61K 45/00; A61K 47/00

(52) U.S. Cl. 424/130.1; 424/531; 424/522; 424/278.1

(57) ABSTRACT

Serum preparations obtained from sharks are used to prepare compositions, including pharmaceutical compositions, for inhibiting retrovirus replication in susceptible cells. Thus, the serum preparations are useful for inhibiting diseases associated with retroviral infection, such as acquired immunodeficiency syndrome. The serum concentrates are also used to inhibit growth of tumor cells, especially sarcomas and leukemias.

### BACKGROUND OF THE INVENTION

The disease acquired immunodeficiency syndrome, or AIDS, remains retractile to therapy. Despite intensive efforts to develop compounds that inhibit the virus that causes the disease, HIV, the infection almost uniformly progresses and the individual's immune system is rendered dysfunctional. The patients become extremely susceptible to secondary diseases, such as pneumonia and Kaposi's sarcoma, and ultimately die. While drugs such as 3′-azido-3′-deoxythymidine (AZT), 2′, 3′-dideoxyinosine (ddI) and 2′, 3′-dideoxycytosine (ddC) have been approved for use in infected individuals, profound toxicities and the emergence of drug-resistant viral strains are associated with their use. There remains a critical need to identify new anti-HIV agents for use alone or in combination with other antiviral agents.

Effective anti-HIV therapy depends on identifying antiviral agents devoid of significant toxicity and which, when employed in therapy, do not readily result in the emergence of drug-resistant viral isolates. Compounds potentially useful in inhibiting HIV retroviral infection are screened in a number of systems. Initially, screening is conducted in in vitro models of susceptible cell lines. Animal models are then used to identify those compounds with anti-HIV activity in vivo as well as possessing acceptable levels of host toxicity. The models preferably assess activity against HIV viremia as well as ability to suppress HIV-induced disease, such as the destruction of the immune system and central nervous system disease.

One model which has been found to be particularly useful as a model of retroviral infection, including HIV infection, is the Rauscher leukemia virus (RLV) infection of mice. RLV is infectious and pathogenic in adult mice, and it is erythrotropic, causing a splenomegaly that is proportional to viral titer. Chirigos, Cancer Res. 24: 1035-1041 (1964). Shortly after inoculation, abnormal spleen colonies are formed whose numbers reflect viral titers. Each colony is the result of a successful viral “hit,” and the colony continues to enlarge while new target cells are being transformed continuously during the viremia. RLV also induces B-cell neoplasms. Thus, RLV infection results in a massive splenomegaly and erythroleukemia which kills infected animals within 4-5 weeks after inoculation. Weiss, Teich, Varmus, et al., “RNA Tumor Viruses,” 2d ed., Cold Spring Harbor, Cold Spring Harbor Laboratory Press, 1984, pp 78-79. RLV infection in mice has also been shown to reproduce certain immunological aspects of HIV infection in humans. See, e.g., Gabrilovich et al., Immunology 82: 82-87 (1994) and Gabrilovich et al., Eur. J. Immunol. 23:2932-2938 (1993).

The use of the RLV model to identify and evaluate anti-HIV agents has become widespread. For example, RLV as a model of HIV infection has been reported for evaluating AZT (Ruprecht et al., Nature 323: 467-469 (1986); Ruprecht, Intervirol. 30(S1): 2-11 (1989)), new lipophilic derivatives of AZT (Schwendener et al., Antiviral Res. 24: 79-93 (1994)), derivatives of tetrahydroimidazole[4,5,1-jk][1,4]-benzodiazepin-2(1H)-thione (Buckheit et al., AIDS Res. Human Retrovir. 9: 1097-1106 (1993), biological response modifiers, including tests conducted by the U.S. Food and Drug Administration, as reported for, e.g., poly [I,C]-LC, MVE-2 and CL 246,738 (Black et al., Annl. N.Y. Acad. Sci. 685: 467-470 (1993)), and combination anti-HIV therapies (e.g., Ruprecht, Buckheit et al., and Black et al., supra). And, because RLV induces a leukemia in infected animals, the RLV model is also used extensively as a model for treating various types of cancers, but particularly leukemias. Sydow and Wunderlich, Cancer Lett. 82: 89-94 (1994).

New therapeutic modalities are urgently needed to provide more effective treatments for HIV infection and the diseases associated with HIV infection. Also needed are effective means to inhibit development of cancers, such as leukemias or other neoplasms. Compositions useful for these purposes should be relatively easy to prepare and administer, relatively non-toxic, and effective inhibitors of retroviral infection or particular neoplasms. The present invention addresses these and other related needs.

### SUMMARY OF THE INVENTION

The present invention provides, in one embodiment, a method for preparing shark serum concentrate that is useful in inhibiting retroviral and neoplastic disease in a susceptible mammalian cell. Generally, the shark serum is produced by an admixture of ethanol and shark blood, followed by removal of the ethanol, typically by heating to a temperature and for a time sufficient to evaporate the ethanol but not sufficient to inactivate serum proteins such as complement. The serum is then separated from any precipitate.

Thus, in one method, ethanol is added to shark blood in an amount sufficient to cause separation of red cells and serum, thereby forming an ethanol-shark blood mixture. Typically the shark blood is obtained frozen and is thawed to, e.g., room temperature, before adding the ethanol. Ethanol may be added in an amount of 20-35% vol/vol, more usually about 25% vol/vol. The ethanol-shark blood mixture is warmed to a temperature, e.g., to about 70-85° F., and for a time sufficient to evaporate the ethanol but not sufficient to inactivate serum complement. The mixture is then separated by centrifugation, filtration or the like into precipitate and shark serum concentrate. The process can then be repeated up to at least three or more times with the serum to achieve further clarify and concentration of immunoglobulin.

In another aspect the invention provides a composition, including pharmaceutical composition, which comprises shark serum produced by a process of admixture of ethanol and shark blood, followed by removal of ethanol, typically by heating but not inactivating serum complement, and separation of serum from any resulting particulate. The composition can be formulated in amounts sufficient to inhibit retroviral replication in susceptible cells or neoplastic disease, and further comprise a pharmaceutically acceptable carrier or stabilizer, such as maltose. The pharmaceutical shark serum composition can also be lyophilized.

In other embodiments the invention provides methods for inhibiting retroviral infection of susceptible mammalian cells. An amount of shark serum sufficient to inhibit or prevent said retroviral infection is administered to the cells. The retrovirus includes the human retroviruses, especially HIV-1 or HIV-2. The shark serum concentrate is administered in a variety of ways, including intravenously, intramuscularly or orally.

In another aspect the invention provides methods for inhibiting tumor cells. Shark serum concentrate is administered to the tumor cells in an amount sufficient to inhibit growth of the tumor. The tumor cells being inhibited can be in a culture, e.g., in vitro, in an afflicted mammal, or removed from the mammal for ex vivo treatment. The tumors susceptible to inhibition include a variety of sarcomas and leukemias, and further include those which are induced by a retroviral gene.

### DESCRIPTION OF THE SPECIFIC EMBODIMENTS

The present invention provides compositions useful in methods for inhibiting or reversing HIV-mediated disease in an infected individual. It has been discovered as part of the present invention that preparations of shark serum inhibit manifestations of retroviral disease in an infected mammal. The shark serum preparations are able to inhibit retroviral titer and associated symptoms and either restore cellular functions or prevent their further deterioration. In other aspects of the invention the shark serum preparations are used to inhibit development of neoplastic disease, such as sarcomas or lymphomas, in afflicted mammals.

As part of the present invention it has been demonstrated that shark serum can be prepared which possesses significant anti-retroviral and anti-neoplastic activity. The shark serum concentrate, when administered to animals infected with the Rauscher murine leukemia virus, inhibits or prevents the development of splenomegaly in the animals in a dose dependent manner, whereas untreated animals develop severe splenomegaly. The Rauscher leukemia virus is a retrovirus which is widely used as an in vivo model of HIV retrovirus infection. A standard measure of drug effectiveness in the Rauscher model has been the ability to inhibit splenomegaly in infected animals. In the experiments described herein the shark serum concentrate completely prevents the splenomegaly observed in Rauscher infected control animals. These findings indicate that symptoms of HIV infection can be inhibited or completely prevented, thereby allowing the immune system of the infected individual to respond more appropriately to other antigens for which an individual's response had been severely depressed, thus extending the life of the individual.

The preparations of shark serum concentrate described herein can be used to treat pathological conditions associated with the retroviral infection, at the cellular level, such as HIV-induced neurological damage, retroviral-induced neoplastic diseases, programmed cell death, and the like. Retroviruses which can be treated by the serum compositions include HTLV-I, HTLV-II, HIV-1, HIV-2, and a variety of animal retroviruses, as exemplified by the Rauscher murine leukemia virus. Dysfunctional processes susceptible to treatment or inhibition by the shark serum concentrate are identified using, e.g., mammalian cells or animals suspected of undergoing the dysfunctional process, e.g., sarcoma cells, which are confirmed to be susceptible to the process, e.g., tumorigenic in the case of the sarcoma cells. To determine that the shark serum concentrate inhibits the dysfunctional process, the cells are treated with the shark serum preparation and the results compared to an untreated cell sample. In affected cells which have been treated with the shark serum concentrate and which demonstrate a inhibition or reversal of a conveniently monitored functional attribute(s), such as neoplastic proliferative capability, the process is determined to be susceptible to treatment according to the present invention. The present methods may also be effective in treating autoimmune or autoimmune-associated diseases, particularly those which are associated with immunodeficiencies as may be associated with HIV infection.

The shark serum concentrate of the present invention is prepared by careful separation of blood components such as hemoglobin and cells from serum components. In a preferred embodiment herein, the blood is initially obtained frozen from a supplier. Shark blood is harvested by a variety of different means. As shark blood begins to clot immediately after death, bleeding must begin as soon as possible. In one preferred method the shark's head is raised to a point higher than the tail, the tail then removed and blood allowed to flow into a suitable container. Blood can also be obtained without sacrificing the shark, such as by employing venipuncture of an anesthetized or immobilized shark, but smaller quantities are obtained. The harvested blood is promptly frozen to prevent deterioration. Blood may be used from a number of different sharks, such as the Mako, Thresher, Black Tip, or Common Reef Shark, but other species would also suffice, e.g., the Sandbar or Nurse sharks. Titers of anti-retroviral and anti-tumor serum activity can be determined by in vitro assays as described herein for a particular species or source. Serum from different species can be pooled, if desired, to achieve a particular level of activity.

To separate the serum from the cellular components the frozen blood is allowed to thaw, typically at room temperature or the like. In a preferred separation and concentration procedure, the thawed blood is admixed with ethanol or suitable alternatives, e.g., fluorinated derivatives of ethanol, at a ratio of about 15-40% ethanol to blood (volume/volume), more preferably 20-35% ethanol, and most preferably in the example described herein a 25% ethanol to blood mixture is used. Pure ethanol (100%) is preferably used in this process. The mixture is gradually brought to a temperature of about 70-90° F. with gentle mixing, more preferably about 70-85° F., and most preferably in a range of about 70-75° F. A heating plate or similar device can be used for this purpose. The temperature will be less than the temperature at which serum complement is inactivated. The mixing should be sufficient to effect a thorough mixture of the blood and alcohol and simultaneously avoiding scalding the preparation at the heating surface. The gentle mixing of the warmed preparation continues until the alcohol has been substantially evaporated from the mixture, generally about 1 to 1 1/2 hours for a 100 ml preparation. The resulting preparation typically will contain a precipitate, and this fraction is separated from the fluid (serum concentrate), typically by centrifugation, filtration or the like. The concentration process may be repeated with the serum concentrate, i.e., repeating the ethanol treatment, heating and mixing, and separating until the resulting shark serum concentrate is clear and the precipitate is virtually eliminated at the final separation step. Typically the process is repeated three to four times to reach the end product. A subsequent test of the final preparation is performed by mixing a sample thereof with ethanol, heating and mixing; the absence of further precipitate indicates a substantially pure serum product. For preparing a pharmaceutically acceptable composition, the shark serum concentrate will typically be sterilized in a manner well known to those familiar with preparing pharmaceutically acceptable serum preparations, e.g., by filtration, irradiation, etc. Methods for preparing pharmaceutically acceptable serum preparations are described in, e.g., U.S. Pat. Nos. 4,587,121, 3,903,262 and 4,186,192, each of which is incorporated herein by reference.

The shark serum concentrate compositions may be administered to persons or mammals suffering from, or predisposed to suffer from HIV associated disease or cancer. The shark serum concentrate is believed to restore functionality, such as immunoproliferative capacity, etc., to HIV-afflicted cells. Thus, not only is replication or spread of the virus impeded by the treatment, but the patient retains or regains a responsive immune system as well and therefore is able to respond to other antigenic challenges and/or to HIV itself. As infections secondary to HIV are a major cause of death of AIDS patients, the treatment afforded by the present invention presents a major step toward eliminating the potentially devastating effects of this disease.

The compositions also find use for pre- or post-exposure prophylaxis, e.g., HIV prophylaxis following dirty needle injuries to health care workers or routinely accompanying blood transfusions or to persons in danger of becoming exposed to infected body or culture fluids. For post-exposure prophylaxis, administration is begun shortly after the suspected inoculation and continues for at least about two to four weeks thereafter, followed by additional dosages or long term maintenance dosages as may be necessary to inhibit growth of the virus and disease and/or to maintain immunity thereto.

The pharmaceutical serum compositions are intended for parenteral, topical, oral, or local administration for prophylactic and/or therapeutic treatment. Preferably, the pharmaceutical compositions are administered orally or parenterally, i.e., intravenously, intraperitoneally, subcutaneously, or intramuscularly. Thus, this invention provides methods which employ compositions for oral or parenteral administration which comprise a solution of a shark serum concentrate in a pharmaceutically acceptable carrier, preferably an aqueous carrier. A variety of aqueous carriers may be used, e.g., water, buffered water, 0.4% saline, 0.3% glycine, and the like, and may include other proteins for enhanced stability, such as albumin, lipoprotein, globulin, etc., subjected to mild chemical modifications or the like. The serum compositions can also be formulated into a cream or salve for topical administration, e.g., at 5-25% concentration. The compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as a pH adjusting and buffering agents, tonicity adjusting agents and the like, for example, sodium acetate, sodium lactate, sodium chloride, potassium chloride, calcium chloride, stabilizers (e.g., maltose (1-20%), etc.

The concentration of the shark serum concentrate in these formulations can vary widely, i.e., from less than about 10%, usually at or at least about 25% to as much as 75 or 90% by weight and will be selected primarily by fluid volumes, viscosities, etc., in accordance with the particular mode of administration selected. Actual methods for preparing orally and parenterally administrable compositions will be known or apparent to those skilled in the art and are described in detail in, for example, Remington's Pharmaceutical Science, 17th ed., Mack Publishing Company, Easton, Pa. (1985), which is incorporated herein by reference.

Determination of an effective amount of shark serum concentrate to inhibit HIV-mediated disease or cancer in a patient can be determined through standard empirical methods which are well known in the art. For example, reversal of impairment of immune function, e.g., restoration of lymphoproliferative response to recall antigen (e.g., influenza), alloantigens or mitogens such as PWM or PHA, and thus efficacy of the subject compositions, can be monitored with a variety of well known in vitro T-cell proliferative response procedures.

Compositions of the invention are administered to a host already suffering from an HIV infection or neoplasm, as described above, in an amount sufficient to prevent or at least partially arrest the development of the ensuing immunodeficiency disease and its complications, or the susceptible tumor as more fully described below. An amount adequate to accomplish this is defined as a “therapeutically effective dose.” Amounts effective for this use will vary considerably and depend on the severity of the infection or disease and the weight and general state of the patient being treated, but generally range from about 5 mg/kg to about 1,000 mg/kg host body weight of shark serum concentrate per application, with dosages of from about 10 mg/kg to about 200 mg/kg of shark serum concentrate per application being more commonly used. Administration can be daily, weekly or less frequently, as necessary depending on the response to the disease and the patient's tolerance of the therapy. Maintenance dosages over a prolonged period of time may be needed, and dosages may be adjusted as necessary. The period of administration will generally be sufficient to restore the immune system of the host, such that effective immune responses can be mounted against a variety of antigens, most desirably the HIV virus in the case of individuals infected with HIV, or to eliminate or substantially inhibit the growth of the cancer cells. If an individual's restored immune system is not able to eliminate the disease, maintenance dosages over a prolonged period may be necessary. Also, it must be kept in mind that the materials of the present invention may be employed in life-threatening or potentially life threatening situations. In such cases, it is possible and may be felt desirable by the treating physician to administer substantial excesses of these compositions. For veterinary uses higher levels may be administered as necessary.

In prophylactic applications, compositions containing the shark serum concentrate are administered to a patient susceptible to or otherwise at risk of retroviral-mediated disease to enhance the patient's own immunologic capabilities. Such an amount is defined to be a “prophylactically effective dose.” In this use, the precise amounts again depend on the patient's state of health and weight, but generally range from about 1 mg/kg to about 250 mg/kg body weight, more commonly from about 10 mg/kg to about 100 mg/kg of body weight.

Single or multiple administrations of the compositions can be carried out with the dose levels and pattern being selected by the treating physician. In any event, the pharmaceutical formulations of the shark serum concentrate should provide a quantity of the inhibitor sufficient to effectively inhibit the retroviral-mediated disease or tumor in the treated host.

The methods of the invention can also be employed for ex vivo therapy. By ex vivo or extracorporeal therapy is meant that therapeutic manipulations are performed on host cells and fluids outside the body. For example, lymphocytes or other target cells may be removed from a patient and treated with high doses of the shark serum concentrate, providing a concentration of inhibitor in the cell far in excess of levels which could be accomplished or tolerated by a patient. Following treatment, the cells are returned to the host to treat the disease.

For use in the present methods a preparation of shark serum concentrate can be combined with one or more other pharmaceutical compositions for a variety of therapeutic uses, e.g., enhanced therapeutic activity against retroviruses such as HTLV-I, HTLV-II, HIV-1 or HIV-2, or cancer. For example, in the treatment of HIV infection, the pharmaceutical compositions of the present invention may be administered alone or as adjunct therapy with, e.g., AZT, ddI, ddC, or combinations thereof, such as AZT, ddI, and shark serum concentrate. When administered as adjunct therapy, the shark serum concentrate can be administered in conjunction with the other treatment modalities, or separately at different intervals.

The shark serum concentrate of the present invention also finds use in vitro. The compositions can be used to inhibit retroviral induced death of cultured cells, such as certain hybridoma or other lymphocyte lines which are susceptible to retroviral infection. The preparations of the invention can also be used in screening assays to assess effective levels of antiretroviral compounds or other treatments. In addition, by determining whether a retrovirus-mediated dysfunction or death of a patient's cells is susceptible to inhibition or reversal by the shark serum concentrate, appropriate therapy can be more readily instituted or, alternatively, the effect of other treatment modalities, such as other anti-HIV regimens, can be determined. Thus, a diagnostic method for assessing the efficacy of, e.g., anti-HIV therapy is also provided by the present invention. Detecting changes in vitro at the level of HIV susceptibility, or such as restoration of immune function, e.g., response to recall antigens, to alloantigens, or to mitogens such as PWM or PHA, provides an indication of in vivo activity of the shark serum concentrate of the present invention.

To monitor changes in the level of immune function in a cell population, control values of immune function may be determined from cells from the general population or from the patient prior to commencement of therapy. Since immune function may vary considerably among patients, determination of each patient's pre-treatment immune function is preferred. The level of immune function in cells, e.g., lymphocytes in the case of HIV-infected individuals, is then monitored during therapy. This level is compared to the level of the immune function in cells not exposed to therapy, and effectiveness of therapy is assessed by an increased level in the measured immune function during or post-therapy.

The shark serum concentrate of the present invention can also be used as an anti-neoplastic agent. Among the neoplastic diseases inhibited by the serum concentrate are sarcomas, leukemias, and carcinomas, including those which may be induced by a retroviral gene. Determination of an effective amount of shark serum concentrate of the invention sufficient to inhibit growth of the neoplastic cells may be determined by, for example, monitoring metastatic sites with a variety of procedures, e.g., in vivo imaging or ex vivo diagnostic techniques. Other cancer markers may also be used to monitor therapy with the serum concentrates of the invention, e.g., the PSA assay for prostate carcinoma, which assay is commercially available.

The therapeutic compositions of the invention are administered to a patient already suffering from a neoplasm, e.g., sarcoma, leukemia or carcinoma, in an amount sufficient to cure or at least partially arrest the disease. An amount adequate to accomplish this is defined as “therapeutically effective dose.” Amounts effective for this use will depend on the severity of the neoplasm and its location, particularly when a metastatic site is implicated, and the weight and general state of the patient being treated, but generally range from about 10 mg/kg to about 1,000 mg/kg host body weight of serum concentrate per day, with dosages of from about 25 mg/kg to about 125 mg/kg per day being more commonly used. Maintenance dosages over a prolonged period of time may be adjusted as necessary. As the shark serum concentrates may be employed in advanced disease states, that is, life-threatening or potentially life threatening neoplastic disease, it is possible and may be felt desirable by the treating physician to administer substantial excesses of these compositions.

Single or multiple administrations of the compositions can be carried out with the dose levels and pattern being selected by the treating physician. In any event, the pharmaceutical formulations should provide a quantity of shark serum concentrate of the invention sufficient to effectively inhibit the neoplastic disease. The pharmaceutical compositions of the present invention may be administered alone or as adjunct therapy. The compositions may be administered with, e.g., taxol, cis-platin, tamoxifen, etoposide phosphate, doxorubicin, daunomycin, endocrine therapy, etc. When administered as adjunct therapy, the compositions of the present invention may be administered in conjunction with the other treatment modalities, or separately at different intervals.

As with methods described herein for inhibiting retroviral-mediated disease, the shark serum preparations of the invention can also be used in ex vivo therapy of neoplastic disease. For example, bone marrow or other target cells or tissues are removed from a patient and treated with high doses of compositions which comprise the shark serum concentrates of the invention, which optionally can be conjugated to cytotoxic agents such as toxins, drugs, labels, etc., proving a therapeutic concentration of the compositions far in excess of levels which could be accomplished or tolerated by the patient. Following treatment to eliminate the neoplastic cells in the target cell population or tissue, the cells or tissues are return to the patient.

The following experimental examples are offered by way of illustration, not by limitation.

### EXAMPLE I

This Example describes the preparation of serum concentrates from shark blood.

Whole shark blood from Mako shark was obtained frozen. Blood was obtained from the sharks by severing the tail and elevating the body, allowing the blood to drain into a container which was sealed and frozen in a refrigerated compartment. The blood was kept frozen until delivered to the laboratory. The frozen blood was allowed to thaw at room temperature.

In a typical preparation, 100 mL of the thawed shark blood was treated with 25% (by volume) pure ethanol at room temperature. The mixture is then brought to a temperature of about 70-75° F., with mixing sufficient to accomplish a thorough mixture of the blood and alcohol and so as to prevent scalding the preparation at the heating surface. The gentle mixing of the warmed preparation continues until the alcohol has been substantially evaporated from the mixture, generally about 1 to 1 1/2 hours for a 100 ml preparation. The resulting preparation contained a precipitate, and this was removed by centrifugation (Physician's Compact Centrifuge, Clay Adams, Inc.) at 1,315×g for 40-45 min. and the precipitate discarded. This process of ethanol treatment, heating and mixing, centrifugation and decanting was repeated three to four times. The resulting fluid is very clear and only a trace of precipitate is obtained at the final centrifugation step. A subsequent test of the final preparation by mixing with ethanol, heating and mixing followed by centrifugation indicates a substantially pure product with no precipitate in the centrifuging vessel.

The original shark blood had a hemoglobin concentration of 16 g per 100 mL and a total protein content of 6.6 g per 100 mL. The shark blood concentrate used in the Rauscher virus inhibition examples described below had a hemoglobin content of 4.6 g per 100 mL and a protein content of 1.5 g per 100 mL.

### EXAMPLE II

This Example describes the use of the serum preparation described in Example I to inhibit manifestations of retrovirus infection in a mammal.

Efficacy of the shark serum preparation as an anti-retroviral agent was determined using the Rauscher virus disease model. Rauscher is a pathogenic murine retrovirus in mice, typically causing erythroid colonies in the spleen of mice leading to a severe splenomegaly, and also causes a erythroleukemia. In this study, BALB/c mice treated with the shark serum preparation and untreated controls were infected with virus preparations.

BALB/c mice weighing 12-15 grams each were used as test animals. The mice were obtained from Harlan Sprague Dawley, Indianapolis, Ind., and were quarantined for a minimum of five days before they were used for testing. The mice were held at stable temperature and observed daily for signs of disease, stress, injury, and external parasites.

The test was conducted as follows. The animals were divided into five groups. Group I, which consisted of twelve animals, served as a control, in that mice were not challenged with Rauscher virus, but were injected IP with 0.2 ml of physiological saline solution daily for five days. Twelve animals of Group 2 were challenged with the Rauscher virus on Day 0 of the study, and received injections IP of physiologic saline solution daily for five days. Group 3A consisted of six animals that were challenged with Rauscher virus on Day 0 and received 0.5 ml shark serum preparation by intraperitoneal injection every day for five days. Group 3B consisted of six animals that were challenged with Rauscher virus on Day 0 and received 0.25 ml shark serum preparation by intraperitoneal injection every day for five days. Group 3C consisted of six animals that were challenged with Rauscher virus on Day 0 and received 0.1 ml shark serum preparation by intraperitoneal injection every day for five days. Each mouse was injected intraperitoneally with 0.1 ml Rauscher leukemia virus infected cells (06-000-000; Advanced Biotechnologies, Inc., Md.). Immediately following virus injection on Day 0, the animals received the first injection of saline or the shark serum preparation. Animals were observed daily for 21 days, and the experiment was terminated on day 21 with the weights of all survivors recorded prior to sacrifice. Spleens were removed from all animals immediately following sacrifice, weighed and recorded.

The results, which can be seen in Table 1, showed that Group 1 animals which received only saline had a mean spleen weight per mouse of 120 mg. Group 2 animals that were challenged with Rauscher virus but received only saline treatment had massive spleens, with a mean spleen weight per mouse of 3,800 mg. These control groups demonstrate the establishment of the retroviral infection and the advancing disease in infected animals. In contrast to the saline treated animals, animals which received 0.5 ml, 0.25 ml, or 0.1 ml of shark serum preparations had dramatically smaller spleens, with mean spleen weights per mouse of 110 mg, 398 mg, and 872 mg, respectively.

TABLE 1

| | Mean Final Body | Mean Spleen Weight |
|---|---|---|
| Group | Wt. Per Mouse (g) | Per Mouse (mg) |
| I (neg. ctrl) | 23.0 | 120 |
| II (pos. ctrl) | 21.5 | 3800 |
| III-A | 22.4 | 110 |
| III-B | 27.3 | 398 |
| III-C | 22.4 | 872 |

The results confirm that the shark serum preparation contained significant anti-retroviral activity and effectively prevented, in the case of the 0.5 ml treatment, or inhibited in the case of the lower doses, the normal progression of retroviral disease in the infected animals.

### EXAMPLE III

This Example demonstrates a significant anti-tumor activity of the shark serum preparation described in Example I.

The tumor used in this study was Sarcoma 180 obtained from the American Type Culture Collection and passed at weekly intervals as an ascites in non-treated female Swiss-Webster mice. To prepare the inoculum of tumor cells for the present study, ascites fluid from a mouse with a 7-10 day ascites was aspirated with sterile technique. The tumor cells were checked for viability using trypan-blue staining. A tumor cell count was then determined, and cells diluted with normal saline or phosphate buffered saline to obtain a final concentration of 1-2×10⁶ cells/mm³. This suspension is used for injection in mice. The final dilution is plated onto trypticase soy agar to determine that it is free from bacterial contamination.

For injection into animals, 0.1 ml of tumor cell suspension is inoculated into the left hind leg muscle of each mouse. Inoculated mice were then placed into a large cage and then randomly segregated into groups of ten mice and then housed under routine conditions. Mice were weighed on the day of inoculation, day 7 and day 14 at the time of sacrifice.

Treatment of the mice was usually initiated one day after injection of the tumor and was continued daily for five days. In one group the treatment was not initiated until day 7, at which time the developing tumor was palpable. In this instance the treatment continued for 5-7 days prior to sacrifice. At the end of the 14 day observation period the mice were sacrificed by cervical dislocation or ether anesthesia. The skin over the left hind leg was removed to expose the tumor, and the leg with tumor was removed at the hip joint. Any residual skin was removed and the leg/tumor was weighed. The mean value of the normal leg was subtracted from the weight of the leg with the tumor to provide an estimate of the actual tumor weight. Percent tumor inhibition was determined as follows:

$$\% Inhibition = \frac{Mean tumor weight ( test )}{Mean tumor weight ( control )} \times 100$$

There were ten mice per group. The test mice were treated on day one (the day after the tumor implant) and continued for five days. Tumor weights were determined on day 14. Ten mice served as controls and were treated with 0.5 ml saline, ten mice received 0.25 ml shark serum preparation, and ten mice received 0.5 ml shark serum preparation. All treatments were administered by intraperitoneal injection.

The results, shown in Table 2, indicated that the shark serum preparation contained significant anti-tumor activity, with mean tumor inhibition of 57.7% and 98.4% for the 0.25 ml and 0.5 ml shark serum preparation treatments, respectively, when compared to tumors of the saline treated control animals.

TABLE 2

| Group | Mean Tumor Wt. (g) | % Inhibition |
|---|---|---|
| Control | 2.43 g. | — |
| 0.25 ml Serum Conc. | 1.03 g. | 57.7% |
| 0.5 ml Serum Conc. | 0.04 g. | 98.4% |

From the foregoing it will be appreciated that, although specific embodiments of the invention have been described herein for purposes of illustration, various modifications may be made without deviating from the spirit and scope of the invention. Accordingly, the invention is not to be limited except as by the appended claims.

What is claimed is:

1. A method for preparing shark serum concentrate useful in inhibiting disease in a mammal, comprising:
(a) adding ethanol to shark blood in an amount sufficient to cause separation of red cells and serum, thereby forming an ethanol-shark blood mixture;
(b) warming the ethanol-shark blood mixture to a temperature and for a time sufficient to evaporate the ethanol but not sufficient to inactivate serum complement; and
(c) separating the mixture into precipitate and shark serum concentrate.

2. The method of claim 1, further comprising the step of repeating steps a-c with the shark serum concentrate.

3. The method of claim 1, wherein the shark blood is obtained frozen and is thawed before adding the ethanol of step (a).

4. The method of claim 1, wherein the ethanol of step (a) is added to shark blood in an amount of 20-35% vol/vol.

5. The method of claim 4, wherein the ethanol is added to shark blood in an amount of about 25% vol/vol.

6. The method of claim 1, wherein the ethanol-shark blood mixture of step (b) is warmed to a temperature of about 70-85° F. while mixing.

7. The method of claim 6, wherein the ethanol-shark blood mixture is maintained at said temperature while mixing for at least about one hour.

8. The method of claim 1, wherein the separation of step (c) is accomplished by centrifugation.

9. The method of claim 1, wherein the separation of step (c) is accomplished by filtration.

10. The method of claim 2, wherein steps (a)-(c) are repeated at least three times.

11. A method for inhibiting retroviral infection of susceptible mammalian cells, which comprises administering to the cells an amount of shark serum sufficient to inhibit said infection.

12. The method of claim 11, wherein the retrovirus is a human retrovirus.

13. The method of claim 12, wherein the human retrovirus is HIV-1.

14. The method of claim 11, wherein the shark serum concentrate is administered to a mammal to inhibit retroviral replication in said mammalian cells.

15. The method of claim 14, wherein the shark serum concentrate is administered intravenously.

16. The method of claim 11, wherein the shark serum is produced by admixture of ethanol and shark blood followed by heating to a temperature and for a time sufficient to remove the ethanol but not sufficient to inactivate serum complement and separation of serum from precipitate.

17. A method for inhibiting tumor cells, which comprises administering to the tumor cells an amount of shark serum concentrate sufficient to inhibit growth of said tumor cells, wherein the serum concentrate is produced by admixture of ethanol and shark blood followed by heating to a temperature and for a time sufficient to evaporate the ethanol but not sufficient to inactivate serum complement and separation of serum from precipitate.

18. The method of claim 17 for inhibiting tumor cells, wherein the tumor cells are contained in an afflicted mammal and the shark serum concentrate is administered to said mammal.

19. The method of claim 18, wherein the tumor is induced by a retroviral gene.

20. The method of claim 18, wherein the tumor is a sarcoma or leukemia.

21. A pharmaceutical composition which comprises shark serum produced by admixture of ethanol and shark blood followed by heating to a temperature and for a time sufficient to evaporate the ethanol but not sufficient to inactivate serum complement and separation of serum from precipitate, in an amount sufficient to inhibit retroviral replication in susceptible cells or neoplastic disease.

22. The pharmaceutical composition of claim 21, further comprising a pharmaceutically acceptable carrier or stabilizer.

23. The pharmaceutical composition of claim 21, wherein the pharmaceutically acceptable stabilizer is maltose.

24. The pharmaceutical composition of claim 21, which is lyophilized.

25. A pharmaceutical composition produced by the method of claim 1.
